(19) United States
(12) Patent Application Publication
(10) Pub. No.: US 2016/0144040 A1
(43) Pub. Date: May 26, 2016

(54) DRUG SUSTAINED RELEASE AGENT BASED ON OLEANOLIC ACID AND A PREPARATION METHOD THEREOF

(71) Applicant: School of Medicine Jiaying University, Meizhou (CN)

(72) Inventor: Jinsheng Cheng, Meizhou (CN)

(21) Appl. No.: 14/948,242

(22) Filed: Nov. 20, 2015

(30) Foreign Application Priority Data
Nov. 26, 2014 (CN) 201410689844.1

(51) Int. Cl. A61K 47/40; A61K 31/19

(52) U.S. Cl. CPC A61K 47/40; A61K 31/19

(57) ABSTRACT

The present invention relates to the technical field of oleanolic acid drugs and provides a drug sustained release agent based on oleanolic acid and a preparation method thereof. The drug sustained release agent based on the oleanolic acid is applied to drugs with the oleanolic acid as a main drug component and is prepared from the components including a drug carrier, a hydrophilic gel material, a erodible matrix material and an insoluble matrix material, wherein the drug carrier is β-cyclodextrin-chitosan composites, wherein the oleanolic acid is from a plant raw material, and a host-guest inclusion complex is composed of the main drug component and the drug carrier according to the mass ratio of 0.1:0.1-0.1:5. A preparation method comprises the following steps: preparing the inclusion complex, mixing auxiliaries, carrying out compression moulding and the like. The drug sustained release agent based on oleanolic acid has the characteristics of stable drug concentration, high biological activity, good drug solubility and long acting effect.

### CROSS-REFERENCE TO RELATED APPLICATIONS

This application is a continuation in part of, and claims priority to, Chinese Patent Application No. 201410689844.1 with a filing date of Nov. 26, 2014. The content of the aforementioned application, including any intervening amendments thereto, is incorporated herein by reference.

### TECHNICAL FIELD

The present invention relates to the technical field of oleanolic acid drugs, more particularly, to a drug sustained release agent based on oleanolic acid and a preparation method thereof.

### BACKGROUND OF THE PRESENT INVENTION

Oleanolic acid is an adjuvant for liver diseases and used for treatment of infectious acute jaundice hepatitis, it is highly effective in reducing glutamic-pyruvic transaminase and removing jaundice and can decline the enzyme, descend the turbid, correct disordered protein metabolism, improve the symptoms of viral and chronic persistent hepatitis patients, promote the liver cell regeneration and prevent cirrhosis and liver ascites; besides, it can also used for psoriasis, rheumatic arthritis, nephritic edema, stomachache, stranguria with turbid discharge, flooding, traumatic injury, swollen welling-abscess, soreness and weakness of waist and knees, threatened abortion and other diseases, so the oleanolic acid has broad application prospects.

The existing oleanolic acid sustained release drugs include capsules, tablets and pills, such as oleanolic acid sustained-release dropping pills, oleanolic acid matrix sustained release tablets, the sustained release preparations are often prepared by the oleanolic acid and sustained release excipient like hydroxypropylmethyl cellose and polyethylene glycol etc. directly. But the oleanolic acid is almost completely insoluble in water, above sustained release methods still exist some limitation in drug loading capacity, water solubility and other aspects, the low water solubility makes the oleanolic acid hard to be absorbed by human body, bioavailability still waits to be increased.

### SUMMARY OF THE PRESENT INVENTION

The present invention solves existing problems of prior art and problems of oleanolic acid drugs like poor dissolution, wide fluctuations of plasma concentration, low bioavailability by preparing a drug sustained release agent based on oleanolic acid, which has the characteristics of drug concentration stability, high biological activity, good drug solubility and long acting effect.

The content of the present invention is as follows.

The drug sustained release agent based on the oleanolic acid is applied to drugs with the oleanolic acid as a main drug component and is prepared from the components including a drug carrier, a hydrophilic gel material, a erodible matrix material and an insoluble matrix material, wherein the drug carrier is a β-cyclodextrin-chitosan composites, wherein the oleanolic acid is from a plant raw material, and a host-guest inclusion complex is composed of the main drug component and the drug carrier according to the mass ratio of 0.1:0.1-0.1:5.

Cyclodextrins are cyclic polysaccharide compounds with 6-12 glucose molecules generated by starch which is under the action of glycosidase of cyclodextrins, the most common cyclodextrins are linked by 6, 7 or 8 glucose molecules through 1,4-glycoside linkage, which is called α-cyclodextrin, β-cyclodextrin and γ-cyclodextrin respectively. As FIG. 1 shown, cyclodextrin is a cone-shaped barrel, a cavity with 0.7-1.0 nm diameter is formed in the middle of cyclodextrin, the inner wall thereof with hydrophobic property is made up of hydrogen atom on 3-C and 5-C in glucose molecules and glycoside oxygen atom, while the outer wall thereof with hydrophilicity is made up of 2-C, 3-C and 6-C terminal hydroxyl group. With the peculiar structure, cyclodextrins can use the hydrophobic cavity to cover guest molecule (material being included) to form inclusion complex by hydrophobic interaction, hydrogen bonding and Van der Waals' force etc. In three kinds of cyclodextrins, β-C cyclodextrin is easier to combine with most drug molecules due to its proper dimension of cavity.

Chitosan contains plenty of hydrophilic groups like hydroxyl and amine on the surface, it has characteristics of natural non-toxicity, good biological compatibility and readily biodegradable etc., the solubility is greatly superior than chitin.

B-cyclodextrin-chitosan composites are cyclodextrin grafted chitosan obtained by the β-cyclodextrin with hydrophobic cavity connected to chitosan through addition, grafting, reductive amination, acylation and other methods, having double characteristics of cyclodextrin and chitosan, the drug loading capacity can be increased through adjusting the content of β-cyclodextrin-chitosan composites, and the diagram of active ingredients is as FIG. 2 shown. The cavity of β-cyclodextrin-chitosan composites has inclusion effect on active pharmaceutical ingredients like oleanolic acid to form a host-guest inclusion complex. Chitosan contains plenty of hydrophilic groups like hydroxyl and amine on the surface, so the solubility of drugs can be increased dramatically and the release of drugs can be delayed. The β-cyclodextrin-chitosan composites are adopted as the drug carrier, so the oleanolic acid and other medicinal ingredients from plant can enter into the cavity of β-cyclodextrin hyperbranched polyamide to form stable host-guest inclusion complex, having higher drug loading capacity; besides, hydrophilic groups of chitosan can increase the solubility of drugs and relieve the release of drugs effectively.

The preparation method of oleanolic acid inclusion complex include methods of precipitation, solution, kneading, grinding, ultrasonic wave, freeze drying or spray drying, different methods can be flexibly adopted as needed.

The plant raw material of oleanolic acid are a mixture of one or more of Chaenomeles sinensis, Chaenomeles speciosa (Sweet) Nakai, Papaya leaves, Micromeria biflora benth, Radix gentianae, Folium eriobotryae, hawthron, Jujube, Celastrus orbiculatus Thunb, Boschniakia rossica, luffa vine, luffa leaves, Clematis manshurica Rupr, Akebia trifoliata, miracle fruit, herba lycopi, Chinese flowering quince's leaves, Achyranthes bidentata, Achyranthes aspera L., Kochia scoparia (L.) Schrad., holly leaf tea, Panax japonicus, Sabia parviflora Wall. ex Roxb, forsythia, roughhaired holly root, Euodia ruticarpa, ginseng, pomegranate peel, Olive-pomace oil, multiflora rose, Radix scrophulariae, Prunella vulgaris, jasmine root, Sambucus chinensis, Eleutherococcus giraldii, purple perilla, Swertia mileensis, Lactuca Indica, Chaenomeles speciosa Nakai, Panax japonicus, Patrinia scabiosaefolia Fisch, Sapindus mukorossi Gaertn, Alyxia sinensis, Common Sowthistle, Osmanthus fragrans (Thunb.) Lour., Sessileflower acanthopanax, Altemanthera philoxeroides (Mart.) Griseb., Phyllanthusnirud Linn, Micromeria biflora benth, Sargentodoxa cuneata, Pogostemon cablin, Ilex rotunda Thunb., fructus corni, Swertia, Ilex cornuta, Eleutherococcus senticosus, plantain, Paulownia tomentosa, Isodon japonica var. galaucocalyx, panax japonicus, Chinese pulsatilla root, Spilanthes acmella, Chinese actinidia root, Vaccinium bracteatum leaves, Patrinia heterophylla, Onychium lucidum, Ficus tsiangii, Taxillus chinensis Danser. Fructus Kochiae Scopariae., Fomes officinalis ames, Patrinia villosa Juss, Radix psammosilenes, Ilex pubilimba, Taxillus chinensis, Glycine soja, Radix rosa laevigata, Alternanthera philoxeroides, Lonicera macranthoides, corn stigma, mint, Acanthopanox trifoliatus (L.)Merr., grape skin, Euphorbia latifolia, Potentilla disclor bunge, Semen celosiae, Anemone flaccida Fr. Schmidt, Clematis brevicaudata, Uncaria macrophylla, Radix Rosa Davuricum, Verbena officinalis, honeysuckle flower, Cornus officinalis, Urena lobata, Mile Swertia Herb, Longan seed, Hypericum monogynum, Lysimachia, phoenix tree flower, Clematis Root, Combretum alfredii, Linearstripe Rabdosia Herb, loquat leaves, mulberry leaves, Ganoderma atrum, Baeckea frutescens, loquat flower, forsythia, indigofera carlesii, Semen Ziziphi Spinosae, memorialis, Rosmarinus officinalis L., Callicarpa macrophylla, Pueraria peduncularis (Grah. ex Benth.) Benth, Acanthopanax giraldii Harms, Osmanthus yunnanensis, Anemone begoniifolia, Sapindus, lilac, Ilex ficoidea, Actinidia chinensis planch, Jasminum lanceolarium, Polygonum cuspidatum, Puberulous Glochidion Herb, Clethra Loosestrife Herb, Viola japonica, Chinese Dicliptera Herb, Nephrolepis auriculata (L.) Trimen, Common Cephalanoplos Herb, Callicarpa peii, Cassia mimosoides Linn., Buddleia, White mulberry Root-bark, Clematis chinensis, Rasberry, mistletoe, Elsholtzia stauntonii Benth, leaves and stems of Panax quinuefolium L and Swertia Punicea. The materials cost can be reduced effectively due to wide sources.

There are unmodified and modified functional β-cyclodextrin-chitosan composites, wherein the modified functional β-cyclodextrin-chitosan composites are hydroxyethyl-β-cyclodextrin-chitosan composites, hydroxylpropyl-β-cyclodextrin-chitosan composites, glucosyl-β-cyclodextrin-chitosan composites, diglucosyl β-cyclodextrin-chitosan composites, carboxymethyl-β-cyclodextrin-chitosan composites, or sulfobutylether-β-cyclodextrin-chitosan composites. Different types of β-cyclodextrin-chitosan composites can be flexibly adopted according to requirements of drug efficacy to reach different sustained release effect.

The hydrophilic gel materials are a mixture of one or more of sodium carboxymethyl cellulose, hydroxypropyl methyl cellulose, calcium alginate, guar gum, chitosan, polyvinyl alcohol, carbopol and DOW polyox water-soluble resin.

The erodible matrix materials are a mixture of one or more of octadecanol, cetyl alcohol, glyceryl behenate, stearic acid, glyceryl monostearate, cholesteryl stearate, carnauba wax, hydroxypropyl methylcellulose phthalate, polymethyl methacrylate, triethyl citrate, glyceryl triacetate and stearic acid.

The insoluble matrix materials are a mixture one or more acrylic resin, polymethyl methacrylate and ethyecellulose.

The auxiliary components are adhesive, excipient, flavoring agent, filler, wetting agent and/or lubricant, wherein the auxiliary components include a mixture of one or more of lactase, starch, polyvinylpyrrolidone, tween, lauryl sodium sulfate, span, lecithin, urea, sucrose ester, polyoxyethylene aliphatate, polyoxyethylene aliphatic alcohol ether, poloxamer, sodium acid carbonate, sodium carbonate and magnesium carbonate.

The sustained release agents can be membrane-controlled release tablets, osmotic pump tablets, matrix tablets, sustained release capsules, sustained release granules or membrane-controlled release pellets. Thereby, the sustained release of drugs is further delayed, plasma concentration is stable and peak valley phenomenon is avoided, besides, relative bioavailability and safety of the active components of oleanolic acid in a human body are enhanced, a dosing frequency is reduced, and compliance of a patient is improved.

A method of the drug sustained release agent based on oleanolic acid, comprising following steps:

The first step: preparing the inclusion complex, the inclusion complex is prepared by oleanolic acid and β-cyclodextrin-chitosan composites according to the mass ratio of 0.1:0.1-0.1:5 by adopting methods of precipitation, solution, kneading, grinding, ultrasonic wave, freeze drying or sprays drying.

The second step: mixing auxiliaries, oleanolic acid drug carrier, β-cyclodextrin-chitosan composites, hydrophilic gel materials, erodible matrix materials, and insoluble matrix materials are weighted respectively according to corresponding technology ratio, and the mixture is mixed sufficiently and evenly.

The third step: carrying out compression moulding, the evenly mixed mixture prepared in the first step is carried out compression moulding by direct compression, granulated compression, pellet compression or coating moulding.

The granulated compression is carried out by dry granulation, wet granulation method or solid phase separation, wherein the coating moulding is carried out by adopting acrylic resin, triethyl citrate, polyethylene glycol, ethyecellulose or cellulose acetate.

The advantages of the present invention are as follows.

Firstly, stable drug concentration, β-cyclodextrin-chitosan composites is used as the drug carrier, meanwhile, the inclusion of small molecule drugs and macromolecular drugs is realized, it is stable released after taking, which is effective to avoid drug concentration fluctuation.

Secondly, high biological activity, cyclodextrins is cyclic polysaccharide compounds with 6-12 glucose molecules generated by starch which is under the action of glycosidase of cyclodextrins, a hydrophilic group is formed on the surface of chitosan, hydrophilic group is connected with pharmaceutical molecules by hydrogen bonding, the composites which is formed by graft of S-cyclodextrin and chitosan have many advantages, such as stability, immunogenicity, no poison, and good biological compatibility;

Thirdly, good drug solubility, the binding force of cyclodextrins, drug molecules and hydrophobic cavity is hydrophobic interaction, hydrogen bonding and Van der Waals' force, and the binding force of chitosan and drug molecules is hydrogen-bond interaction, so it is convenient for drugs to transport after taking and increase the solubility of drugs.

Fourthly, long acting effect. The release process of drug molecule is slow due to a certain volume of hydrophobic cavity inside the cyclodextrin and more parent groups of chitosan, which is effective to prolong the duration time of drugs.

Fifthly, low cost, it is convenient for mass production due to simple preparation method and wide materials origin of the sustained release agents, so the production cost of drug sustained release agents are reduced.

### BRIEF DESCRIPTION OF THE DRAWINGS

FIG. 1 is a stereochemical structure diagram of cyclodextrin.

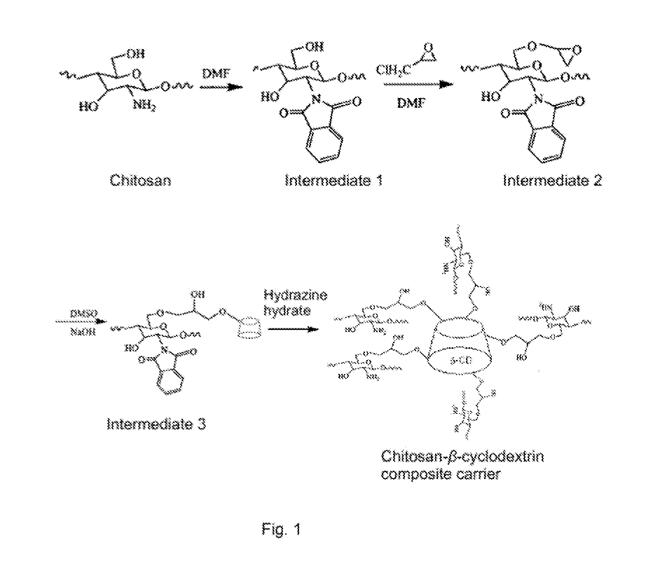

FIG. 2 is a diagram of β-cyclodextrin-chitosan composites covered active ingredients of oleanolic acid.

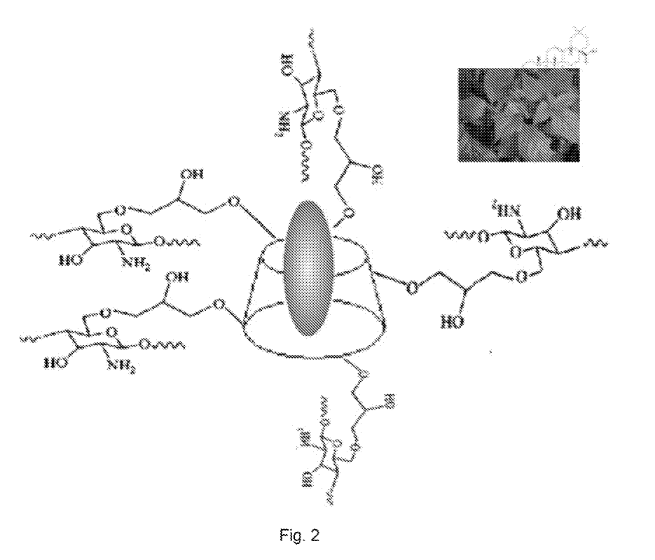

### DETAILED DESCRIPTION OF PREFERRED EMBODIMENTS

For better understanding of the present invention, the following is detailed description about the content and embodiments of the present invention with reference to drawings.

A method of the drug sustained release agent based on oleanolic acid, comprising following steps:

The first step: preparing the inclusion complex, the inclusion complex is prepared by oleanolic acid and drug carrier according to the mass ratio of 0.1:0.1-0.1:5 by adopting methods of precipitation, solution, kneading, grinding, ultrasonic wave, freeze drying or spray drying, wherein the drug carrier is β-cyclodextrin-chitosan composites.

There are unmodified and modified functional β-cyclodextrin-chitosan composites.

The modified functional β-cyclodextrin-chitosan composites are hydroxyethyl-β-cyclodextrin-chitosan composites, hydroxylpropyl-β-cyclodextrin-chitosan composites, glucosyl-β-cyclodextrin-chitosan composites, diglucosyl β-cyclodextrin-chitosan composites, carboxymethyl-β-cyclodextrin-chitosan composites, or sulfobutylether-β-cyclodextrin-chitosan composites.

The second step: mixing auxiliaries, oleanolic acid drug carrier, β-cyclodextrin-chitosan composites, hydrophilic gel materials, erodible matrix materials, and insoluble matrix materials are weighted respectively according to corresponding technology ratio, and the mixture is mixed sufficiently and evenly.

The hydrophilic gel materials are a mixture of one or more of sodium carboxymethyl cellulose, hydroxypropyl methyl cellulose, calcium alginate, guar gum, chitosan, polyvinyl alcohol, carbopol and DOW polyox water-soluble resin.

The erodible matrix materials are a mixture of one or more of octadecanol, cetyl alcohol, glyceryl behenate, stearic acid, glyceryl monostearate, cholesteryl stearate, carnauba wax, hydroxypropyl methylcellulose phthalate, polymethyl methacrylate, triethyl citrate, glyceryl triacetate and stearic acid.

The insoluble matrix materials are a mixture of one or more of acrylic resin, polymethyl methacrylate and ethyecellulose. The auxiliary components are adhesive, excipient, flavoring agent, filler, wetting agent and/or lubricant, wherein the auxiliary components include a mixture of one or more of lactase, starch, polyvinylpyrrolidone, tween, lauryl sodium sulfate, span, lecithin, urea, sucrose ester, polyoxyethylene aliphatate, polyoxyethylene aliphatic alcohol ether, poloxamer, sodium acid carbonate, sodium carbonate and magnesium carbonate.

The third step: carrying out compression moulding, the evenly mixed mixture prepared in the first step is carried out compression moulding by direct compression, granulated compression, pellet compression or coating moulding.

The sustained release agents can be membrane-controlled release tablets, osmotic pump tablets, matrix tablets, sustained release capsules, sustained release granules or membrane-controlled release pellets.

The granulated compression is carried out by dry granulation, wet granulation method or solid phase separation, wherein the coating moulding is carried out by adopting acrylic resin, triethyl citrate, polyethylene glycol, ethyecellulose or cellulose acetate.

To further explain the effect of drug sustained release agent of present invention, a drug sustained release agent based on oleanolic acid of embodiments 1˜3 is prepared according to the preparation method of the present invention, and the drug release is tested, meanwhile, a release curve is made for the drug sustained release agent of embodiment 1, see table 1 and table 2 for details.

TABLE 1
Main Ratio for drug sustained release
agent of different embodiments

| | Main | |
|---|---|---|
| No. | Ingredients | Ratio |
| Embodiment 1 | Inclusion | 41.6 g(including 6 g oleanolic acid and |
| | complex | 35 g β-cyclodextrin-chitosan) |
| Embodiment 2 | Inclusion | 46.6 g(including 6 g oleanolic acid and |
| | complex | 40 g hydroxylpropyl-β-cyclodextrin- |
| | | chitosan) |
| Embodiment 3 | Inclusion | 33.6 g(including 6 g oleanolic acid and |
| | complex | 36 g diglucosyl-β-cyclodextrin-chitosan) |

The oleanolic acid inclusion complex is prepared by solution method in embodiment 1, grinding method in embodiment 2 and precipitation method in embodiment 3 respectively, all the mass of which is dosage of preparing 1000 tablets drug sustained release agent.

TABLE 2
Drug Concentration Release Data of Drug
Sustained Release Agent with Time

| | Sampling point | | | | | |
|---|---|---|---|---|---|---|
| No. | 2 h | 4 h | 6 h | 12 h | 18 h | 24 h |
| Embodiment 1 | 30.88% | 45.22% | 52.82% | 78.05% | 89.53% | 99.38% |
| Embodiment 2 | 29.70% | 38.63% | 50.95% | 76.16% | 88.41% | 98.26% |
| Embodiment 3 | 27.32% | 39.77% | 42.35% | 76.28% | 89.11% | 98.86% |

As table two shown, the drug efficacy of drug sustained release agent based on oleanolic acid of the present invention can last 24 hours, which is long duration; the release concentration is first quick and back slow, and the speed slow down gradually with certain concentration gradient, the change of drug concentration is stable.

The above disclosure merely shows several specific embodiments of the present invention, and the present invention is not limited thereto; those ordinary skilled in the art complete the implementation of the present invention without difficulty based on the drawings of description and above disclosure; while it should be noted to those skilled in the art that several variations, modification and improvements can also be made within the scope of technical proposal, and these variations, modification and improvements are equivalent embodiments; moreover, they are also considered within the protective scope of the present invention.

I claim:

1. The drug sustained release agent based on the oleanolic acid is applied to drugs with the oleanolic acid as a main drug component and is prepared from the components including a drug carrier, a hydrophilic gel material, a erodible matrix material and an insoluble matrix material, wherein the drug carrier is β-cyclodextrin-chitosan composites, wherein the oleanolic acid is from a plant raw material, and a host-guest inclusion complex is composed of the main drug component and the drug carrier according to the mass ratio of 0.1:0.1-0.1:5.

2. A drug sustained release agent based on oleanolic acid according to claim 1, characterized in that there are unmodified and modified functional β-cyclodextrin-chitosan composites, wherein the modified functional β-cyclodextrin-chitosan composites are hydroxyethyl-β-cyclodextrin-chitosan composites, hydroxylpropyl-β-cyclodextrin-chitosan composites, glucosyl-β-cyclodextrin-chitosan composites, diglucosyl β-cyclodextrin-chitosan composites, carboxymethyl-β-cyclodextrin-chitosan composites, or sulfobutylether-β-cyclodextrin-chitosan composites.

3. A drug sustained release agent based on oleanolic acid according to claim 1, characterized in that the plant raw materials of oleanolic acid are a mixture of one or more of Chaenomeles sinensis, Chaenomeles speciosa (Sweet) Nakai, Papaya leaves, Micromeria biflora benth, radix gentianae, Folium eriobotryae, hawthron, Jujube, Celastrus orbiculatus Thunb, Boschniakia rossica, luffa vine, luffa leaves, Clematis manshurica Rupr, Akebia trifoliata, miracle fruit, herba lycopi, Chinese flowering quince's leaves, Achyranthes bidentata, Achyranthes aspera L., Kochia scoparia (L.) Schrad., holly leaf tea, Panax japonicus, Sabia parviflora Wall. ex Roxb, forsythia, roughhaired holly root, Euodia ruticarpa, ginseng, pomegranate peel, Olive-pomace oil, multiflora rose, Radix scrophulariae, Prunella vulgaris, jasmine root, Sambucus chinensis, Eleutherococcus giraldii, purple perilla, Swertia mileensis, Lactuca Indica, Chaenomeles speciosa Nakai, panax japonicus, Patrinia scabiosaefolia Fisch, Sapindus mukorossi Gaertn, Alyxia sinensis, Common Sowthistle, Osmanthus fragrans (Thunb.) Lour., Sessileflower acanthopanax, Alternanthera philoxeroides (Mart.) Griseb., Phyllanthusnirud Linn, Micromeria biflora benth, Sargentodoxa cuneata, Pogostemon cablin, Ilex rotunda Thunb., Fructus corni, Swertia, Ilex cornuta, Eleutherococcus senticosus, plantain, Paulownia tomentosa, Isodon japonica var. galaucocalyx, Panax japonicus, Chinese pulsatilla root, Spilanthes acmella, Chinese actinidia root, vaccinium bracteatum leaves, Patrinia heterophylla, Onychium lucidum, Ficus tsiangii, Taxillus chinensis Danser, Fructus Kochiae Scopariae., fomes officinalis ames, Patrinia villosa Juss, Radix psammosilenes, Ilex pubilimba, Taxillus chinensis, Glycine soja, Radix rosa laevigata, Alternanthera philoxeroides, Lonicera macranthoides, corn stigma, mint, Acanthopanox trifoliatus (L.)Merr., grape skin, Euphorbia latifolia, Potentilla disclor bunge, Semen celosiae, Anemone flaccida Fr. Schmidt, Clematis brevicaudata, Uncaria macrophylla, Radix Rosa Davuricum, Verbena officinalis, honeysuckle flower, Cornus officinalis, Urena lobata, Mile Swertia Herb, Longan seed, Hypericum monogynum, Lysimachia, phoenix tree flower, Clematis Root, Combretum alfredii, Linearstripe Rabdosia Herb, loquat leaves, mulberry leaves, Ganoderma atrum, Baeckea frutescens, loquat flower, forsythia, indigofera carlesii, Semen Ziziphi Spinosae, memorialis, Rosmarinus offcinalis L., Callicarpa macrophylla, Pueraria peduncularis (Grah. ex Benth.) Benth, Acanthopanax giraldii Harms, Osmanthus yunnanensis, Anemone begoniifolia, Sapindus, lilac, Ilex ficoidea, Actinidia chinensis planch, jasminum lanceolarium, polygonum cuspidatum, Puberulous Glochidion Herb, Clethra Loosestrife Herb, Viola japonica, Chinese Dicliptera Herb, Nephrolepis auriculata (L.) Trimen, Common Cephalanoplos Herb, Callicarpa peii, Cassia mimosoides Linn., Buddleia, White mulberry Root-bark, Clematis chinensis, rasberry, mistletoe, Elsholtzia stauntonii Benth, leaves and stems of Panax quinuefolium L and Swertia Punicea.

4. A drug sustained release agent based on oleanolic acid and a preparation method thereof according to claim 2, characterized in that the hydrophilic gel materials are a mixture of one or more of sodium carboxymethyl cellulose, hydroxypropyl methyl cellulose, calcium alginate, guar gum, chitosan, polyvinyl alcohol, carbopol and DOW polyox water-soluble resin.

5. A drug sustained release agent based on oleanolic acid and a preparation method thereof according to claim 2, characterized in that the erodible matrix materials are a mixture of one or more of octadecanol, cetyl alcohol, glyceryl behenate, stearic acid, glyceryl monostearate, cholesteryl stearate, carnauba wax, hydroxypropyl methylcellulose phthalate, polymethyl methacrylate, triethyl citrate, glyceryl triacetate and stearic acid.

6. A drug sustained release agent based on oleanolic acid and a preparation method thereof according to claim 2, characterized in that the insoluble matrix materials are a mixture of one or more of acrylic resin, polymethyl methacrylate and ethyecellulose.

7. A drug sustained release agent based on oleanolic acid according to claim 4, characterized in that the drug sustained release agents further comprise auxiliary components, wherein the auxiliary components are adhesive, excipient, flavoring agent, filler, wetting agent and/or lubricant, wherein the auxiliary components include a mixture of one or more of lactase, starch, polyvinylpyrrolidone, tween, lauryl sodium sulfate, span, lecithin, urea, sucrose ester, polyoxyethylene aliphatate, polyoxyethylene aliphatic alcohol ether, poloxamer, sodium acid carbonate, sodium carbonate and basic magnesium carbonate.

8. A drug sustained release agent based on oleanolic acid according to claim 5, characterized in that the sustained release agents can be membrane-controlled release tablets, osmotic pump tablets, matrix tablets, sustained release capsules, sustained release granules or membrane-controlled release pellets.

9. A preparation method of the drug sustained release agent based on oleanolic acid according to claim 1, characterized in that it comprises following steps:
The first step: preparing the inclusion complex, the inclusion complex is prepared by oleanolic acid and β-cyclodextrin-chitosan composites according to the mass ratio of 0.1:0.1-0.1:5 by adopting methods of precipitation, solution, kneading, grinding, ultrasonic wave, freeze drying or sprays drying.
The second step: mixing auxiliaries, oleanolic acid drug carrier, β-cyclodextrin-chitosan composites, hydrophilic gel materials, erodible matrix materials, and insoluble matrix materials are weighted respectively according to corresponding technology ratio, and the mixture is mixed sufficiently and evenly.
The third step: carrying out compression moulding, the evenly mixed mixture prepared in the first step is carried out compression moulding by direct compression, granulated compression, pellet compression or coating moulding.

10. A preparation method of the drug sustained release agent based on oleanolic acid according to claim 9, characterized in that the granulated compression is carried out by dry granulation, wet granulation method or solid phase separation, wherein the coating moulding is carried out by adopting acrylic resin, triethyl citrate, polyethylene glycol, ethyecellulose or cellulose acetate.

11. A drug sustained release agent based on oleanolic acid and a preparation method thereof according to claim 3, characterized in that the insoluble matrix materials are a mixture of one or more of acrylic resin, polymethyl methacrylate and ethyecellulose.

12. A drug sustained release agent based on oleanolic acid and a preparation method thereof according to claim 3, characterized in that the hydrophilic gel materials are a mixture of one or more of sodium carboxymethyl cellulose, hydroxypropyl methyl cellulose, calcium alginate, guar gum, chitosan, polyvinyl alcohol, carbopol and DOW polyox water-soluble resin.

13. A drug sustained release agent based on oleanolic acid and a preparation method thereof according to claim 3, characterized in that the erodible matrix materials are a mixture of one or more of octadecanol, cetyl alcohol, glyceryl behenate, stearic acid, glyceryl monostearate, cholesteryl stearate, carnauba wax, hydroxypropyl methylcellulose phthalate, polymethyl methacrylate, triethyl citrate, glyceryl triacetate and stearic acid.
